(19) United States
(12) Patent Application Publication
(10) Pub. No.: US 2008/0312344 A1
(43) Pub. Date: Dec. 18, 2008

(54) DENDRIMERS MULTIVALENTLY SUBSTITUTED WITH ACTIVE GROUPS

(76) Inventors: Robertus Matthias J. Liskamp, Utrecht (NL); Dirk Thomas S. Rijkers, Eindhoven (NL); Roelof Jan Pieters, Utrecht (NL); Arwin John Brouwer, Utrecht (NL); Johannes Albertus, F. Joosten, Gendt (NL)

Correspondence Address: BANNER & WITCOFF, LTD., 28 STATE STREET, 28th FLOOR, BOSTON, MA, 02109-9601, US

(21) Appl. No.: 11/956,719

(22) Filed: Dec. 14, 2007

Related U.S. Application Data
(63) Continuation of application No. PCT/NL2006/000295, filed on Jun. 16, 2006.

(30) Foreign Application Priority Data
Jun. 17, 2005 (EP) EP 05076421.6

(51) Int. Cl. A61K 47/48; A61K 47/34

(52) U.S. Cl. 514/772.3

(57) ABSTRACT

The present invention relates to dendrimers being multivalently substituted with active groups, as well as to processes for the preparation thereof and to the use of such multivalent dendrimers.

The active compounds encompass peptides, including oligopeptides up to entire proteins, carbohydrates and pharmaceutically active drug molecules.

### RELATED APPLICATIONS

This application is a continuation of PCT application no. PCT/NL2006/000295, designating the United States and filed Jun. 16, 2006; which claims the benefit of the filing date of European application no. EP 05076421.6, filed Jun. 17, 2005; each of which is hereby incorporated herein by reference in its entirety for all purposes.

### FIELD

The present invention relates to dendrimers multivalently substituted with active groups, and especially with biologically active groups or with diagnostically usable groups. In addition, the present invention relates to a process for substituting particular dendrimers with said active groups. Further, the present invention relates to intermediates for these multivalent dendrimers. In yet a further aspect, the present invention relates to the use of the multivalent dendrimers of the invention.

### BACKGROUND

In the art, and especially in the pharmaceutical field or in the field of diagnosis, there is a need to have available compounds containing a number of active moieties and particularly biologically active moieties. One compound containing a number of active moieties and preferably more than two of such active moieties, which preferably are the same active moiety, is called in the present description and appending claims a “multivalent” compound. Multivalent compounds lead to an increase in activity, and especially in biological or systemic activity or diagnostic efficiency. More in particular, multivalency is an important principle in nature to increase weak interactions, e.g. between ligands and receptors, to biologically important levels.

### SUMMARY

The present invention is especially directed to dendrimers. As the skilled person knows, dendrimers are molecules that at least partially consist of repeating units, and which are molecules that have a two- or three-dimensional diverting branching structure starting from the “base” of the dendrimer. The name is derived from the Greek word for tree; dendrimers are (often) tree-shaped molecules.

The “base” or “core” of the dendrimer was the starting point for its assembly in the chemical construction. Where the assembly of the dendrimer skeleton ends, the “periphery” or “surface” of the dendrimer, a number of active groups are substituted leading to a multivalent dendrimer.

Dendrimers are molecular scaffolds for increasing effects merely by offering a number of ligands or by aligning these ligands. A crucial issue is the complete and efficient attachment of ligands to dendrimers. In the prior art, there are only a few possibilities of attaching peptides to complex molecules such as dendrimers; and even less possibilities to attach unprotected peptides to complex molecules such as dendrimers, let alone possibilities usable at a reproducible an industrial scale. In most cases peptides are attached to dendrimers by chemoselective reaction of sulfhydryl groups of peptides, and especially of the cysteine moieties thereof, with maleimide or iodoacetamide functionalities. However, new reactions with increased efficiency and chemoselectivity (preferably at physiological conditions) would be very welcome.

Srinivasachar et al. point in Abstracts of Papers Part 1 of the 227^{th} ACS National Meeting (0-8412-3924-X) of the American Chemical Society (Anaheim, Calif., USA, March 28-Apr. 1, 2004) to a promise for use of synthetic dendrimers applications. Although it is stated that 1,3-dipolar cycloaddition reactions of sugar azides with various alkyne dendrons (not dendrimers) are being examined to yield regioselective triazole-containing dendrimers with peripheral amine groups, it is unclear how such dendrimers can be formed from sugar azides.

Chitaboina et al. describe in Tetrahedron Letters (2005) 46:2331-2336 a one-pot synthesis of triazole-linked glycoconjugates. In Table 3 regular glycoconjugates not glycodendrimers are shown. Such compounds have not the required “at least partially repeating units” characteristic for dendrimers of the present invention.

Wu et al. in Angew. Chem. Int. Ed. (2004) 43:3928-3932 describes the manufacture of dendrimers. No active compounds are attached to the periphery thereof.

In WO-A-00/16807 and in Sheldon et al. Proc. Nat. Acad. Sci. USA (1995) 92:2056-2060 dendrimers consisting of amino acid units are described. However, none of these known dendrimers contain the azide or alkyn moieties, allowing to couple (biologically) active compounds to the periphery through heterocyclic bridges.

Particularly, there is a need for a multivalent display of active compounds, particularly biologically active compounds and especially a multivalent display of peptides and carbohydrates, in order to increase the affinity of binding to e.g. bacteria, bacterial toxins, enzymes, receptors, antigens or antibodies, etc.

It is an object of the present invention to provide multivalent dendrimers which are well-defined.

It is another object of the present invention to provide homodisperse multivalent dendrimers.

It is yet another object of the present invention to provide processes to prepare the dendrimers of the objects defined in the previous paragraphs, especially in a convenient and preferably efficient way. This especially to overcome the problems of the prior art methods that vary in efficiency and compatibility with functional groups, and which known methods tend to yield all kinds of side-products which are undesirable in couplings involving very precious compounds.

It is yet another object to provide a process which allows a flexible method of introducing a wide variety of active compounds in the multivalent dendrimers.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 schematically depicts a multivalent dendrimer according to the invention.

FIG. 2 depicts second, third, and fourth generation dendrimers 3, 4, and 5, respectively.

FIGS. 3A-3E depict compounds 3G, 4G and 6-31. (A) 1G and 2G; (B) 3G; (C) 4G; (D) 6-11, 15-25; (E) 12-14, 26-31.

FIG. 4 depicts compounds 32-40.

FIG. 5 depicts compounds 41-47.

FIGS. 6A-6B schematically (A) and chemically (B) depicts how an alkyne moiety on the probe allows for “click chemistry” to introduce a fluorescent label for visualization.

FIG. 7 depicts fluorescent gel images obtained of the probe for galectin-3.

FIG. 8 depicts fluorescent gel images obtained of the probe for galectin-3 after contacting bacterial lysate.

### DETAILED DESCRIPTION OF CERTAIN PREFERRED EMBODIMENTS

The present invention aims to solve at least one, preferably more, most preferably all of the foregoing objects.

In a first aspect, the present invention relates to a multivalent dendrimer, comprising at the dendrimer periphery a number of active compounds, and especially of biologically active compounds, the active compounds being coupled to the dendrimer through a 1,2,3-triazole or 1,2-oxazole aromatic heterocyclic bridge. Hence, as active compound according to this description and the appending claims is a compound having a functional group for attachment or conversion to a moiety suitable for the formation of such a heterocyclic bridge, while its structure to achieve a particular activity, such as the binding to receptors etc. is, at least partially, maintained.

Examples of active compounds are drugs that is any drug molecule having a systemic effect after attachment to the dendrimeric structure. Active compounds based on peptides; carbohydrates, such as galactose and other sugar moieties; and drugs such as antibiotics, zanamivir or tamiflu may be attached to the dendrimers of the invention.

The multivalent dendrimers of the invention are versatile constructs for the simultaneous presentation of especially biologically relevant ligands. These multivalent constructs are especially interesting for enhancing the interaction of weakly interacting individual ligands e.g. carbohydrates.

This aromatic heterocyclic bridge may consist only of the aromatic heterocyclic linking group, but may additionally contain one or more spacer groups.

The nature of the spacer or linking group is not critical, as long as it does not adversely interfere with the intended end use of the final multivalent dendrimers of the invention. Actually, the use of spacer groups may have an advantage in that the active compounds, for instance compounds that are recognized by receptors in a system such as an animal or human body, have more flexibility to be able to bind to one or more of such receptors without being hindered by the dendrimeric starting structure. Examples of suitable spacers are for instance hydrophilic spacers, such as those consisting of straight or branched C₁₋₆ alkylene groups, such as ethylene or propylene moieties arranged between two hydrophilic groups such as oxo, thio, amino, amide or ureido groups.

Although it is possible to achieve some of the objects of the present invention, when the dendrimer only contains two or three branches (a first generation dendrimer), it is preferred that the dendrimer is at least a second generation dendrimer, preferably at least a third or fourth generation dendrimer. A second generation dendrimer is a dendrimer wherein the branches derived from the repeating unit (or monomer) defining the dendrimer are coupled (optionally through a linking group or a spacer) to a further repeating unit. For the third and further generation these further repeating units are subsequently coupled to further repeating units.

The number of active compounds coupled to the periphery of the starting dendrimer is at least 2, but preferably higher. Particularly, multivalent dendrimers encompass, but are not limited to, di-, tri-, tetra-, octa-, nona- and hexadecavalent dendrimers. By combining two or more monomers, almost all possible valencies are achievable. For instance, if a monomer having two arms and a monomer having three arms are attached to a monomer having two arms, a pentavalent system is obtained. Starting from and using only monomers having three arms a second generation will have 9, a third generation will have 27 and a fourth generation will have 81 valencies.

Many dendrimers known in the prior art are heterodisperse, because they are mixtures of different molecules. Particularly, during their formation defects occur, because in the respective syntheses, it is very difficult to achieve complete reactions especially in higher generations.

These known heterodisperse dendrimers can form the basis for the multivalent dendrimers according to the invention. However, in accordance with the present invention also homodisperse multivalent dendrimers may be provided, these homodisperse multivalent dendrimers forming a preferred embodiment of the present invention. In a further preferred embodiment, the dendrimer forming the basis of the presently described multivalent dendrimers is amino acid based. Such amino acid based dendrimers provide the possibility of being homodisperse, and in addition are easily preparable.

Amino acid based dendrimers are known in the art and can for instance be synthesized by the method described by Brouwer et al. in Eur. J. Org. Chem. (2001), pp. 1903-1915. Particularly, a reliable and efficient method is described in said reference using amino acids as repeating unit, which amino acids contain at least two amino groups and one carboxyl acid group or at least two carboxylic acid groups and one amino group. The remainder of these amino acids is not critical, as long as the reaction between the amino and the carboxylic acid groups is not disturbed. Particularly, dendrimers can be composed of natural aminoacids, for example those present in proteins. Suitable examples are lysine, diaminobutyric acid, and diaminopropionic acid. However, also other (synthetic) amino acids can be used. Amino acids useful in the dendrimers of the present invention may e.g. comprise an aromatic nucleus providing rigidity or pre-organization to the ultimate dendrimer, which is believed beneficial for its activity. An example of such a monomer is 3,5-di-(2-aminoethoxy)-benzoic acid. However, also the other monomers taught in said Brouwer et al. reference can be suitably used.

In the multivalent dendrimer according to the invention, each active compound coupled to the periphery of the dendrimer is preferably selected from the group consisting of peptides, carbohydrates or drugs.

Especially when the active compounds are the same, that is, represent the same group, very useful dendrimers are obtained. This, however, does not mean that when the active compounds do not have the same meaning, a less suitable multivalent dendrimer is obtained. If, for instance a number of different peptide fragments of proteins expressed by disease causing, or otherwise undesired bacteria or viruses are attached to the dendrimer of the invention, a synthetic vaccine or an antibacterial or antiviral composition may be created.

In a further preferred embodiment, the multivalent dendrimer according to the invention, comprises a core that is substituted by another active compound than the active compounds at the periphery.

The multivalent dendrimers of the present invention will be described in more detail, herein-below.

In a second major aspect, the invention relates to a process for the preparation of the multivalent dendrimers according to the invention, which process comprises reacting a dendrimer having a number of alkyne moieties at its periphery with active compounds comprising an azide, nitrile oxide or nitrone moiety, or reacting a dendrimer having a number of azide or nitrile moieties at its periphery with active compounds comprising an alkyne moiety.

One of the major advantages of the present invention is that the core of the dendrimer is available for a substitution different from the active compounds. A further important aspect is that by using suitable dendrimer building units the exact number of valencies can be controlled in such a way that optimal (binding) activity can be achieved for the multivalent active groups.

This process is based on the cycloaddition reaction between an acetylene and either an azide or an nitrile oxide, leading to a 1,4-disubstituted 1,2,3-triazole or a 1,4-disubstituted 1,2-oxazole bridging unit. This cycloaddition reaction, especially leading to an 1,2,3-triazole, is also referred to as the “click” reaction. The click reaction between the azide and acetylene is known as the Huisgen cycloaddition and is catalyzed by Cu(I) compounds.

The present inventors found that this click reaction is very suitable for chemoselective reactions on dendrimers, and especially with an aim of introducing multivalent peptide and carbohydrate active groups.

Recently, this click reaction, also in combination with microwave, has been described for a number of different reactions. See for instance Savin et al. in Molecular Diversity (2003) 7:171-174. However, this reaction has not been described to prepare multivalent dendrimers wherein the potential click sites are completely converted to 1,2,3-triazole or 1,2-oxazole bridges. Click reactions known to the present inventors generally are used to form one single bridge, or alternatively for non-complex reaction systems.

Especially when the reaction is microwave assisted, a very effective and efficient preparation process is obtained. The use of a microwave results in an efficient and short heating, which heating step is especially effective in the coupling of peptides and carbohydrates to the dendrimer. It effects a very high, and even complete conversion of the alkyne or alternatively the azide or nitrile oxide moieties present at the periphery of the dendrimer. This results in a defined and reproducible multivalent dendrimer product, which is essential to prepare such multivalent dendrimers for pharmaceutical and diagnostical purposes on a practically useful scale.

The microwave assisted click reaction is so quick and selective, that in many cases the active compound or group to be coupled to the periphery of the dendrimer need not be protected. When active groups, such as peptides and carbohydrates, are used in an unprotected form, a deprotecting step is not required. This results in a much simpler process. No interference occurs with other groups than the essential groups for the click reaction.

Because the—preferably-amino acid based dendrimers having a number of alkyne moieties at its periphery, or having a number of azide or nitrile oxide moieties at its periphery have, for as far as the inventors know, not been described in the prior art, these intermediates in the process of preparing the multivalent dendrimers of the invention also form part of this invention.

Yet another aspect of the invention relates to the use of the multivalent dendrimers described in the present description or made by the process described herein, as a pharmaceutical agent or as a diagnostic agent.

Now, the invention will be described in more detail.

As said, the dendrimers used as starting compound in the present invention contain particular surface groups which are able to form a covalent bond via cycloaddition to the active group forming moieties. Particularly, the cycloaddition reaction used in the present invention leads to the creation of 1,2,3-triazole or 1,2-oxazole aromatic heterocyclic bridges between the dendrimer and the active compound. Such bridges are formed by the click reaction between an alkyne and an azide or between an alkyne and on nitrile oxide moiety. It is possible to have the alkyne or acetylene moiety present as a substituent attached to the active compound and the azide or nitrile oxide moiety present as substituent of the dendrimer; it is however preferred to have the alkyne or acetylene moiety present as substituent on the dendrimer, and the azide or nitrile oxide group as substituent attached to the active compound.

The dendrimers used as starting compound in the present invention contain alkyne, azide or nitrile oxide moieties at the periphery or surface. These moieties can be introduced after completion of the (known) dendrimer synthesis or alternatively by incorporation as part of the last peripheral or surface-dendrimer monomer used for construction of the dendrimer.

Preferably, the dendrimers used are amino acid based dendrimers. One of the reasons as to why the amino acid based dendrimers are preferred is that amino groups can easily be converted in azido groups or alternatively acetylene/alkyne groups can be attached. The former is preferred when the active compound to be attached has an acetylene or alkyne substitution. Moreover, azide or acetylene group containing dendrimer building units can easily be synthesized.

The same reason that an amino group can be easily converted in an azido group, makes that all sorts of peptides, ranging from oligopeptides up to entire proteins, can be clicked to dendrimers containing acetylene groups at their periphery. Not only can the N-terminus of each peptide serve as a “handle” for the click reaction, but also each amino acid residue containing an amino group in the side chain, such as a lysine residue or such a side group in synthetic amino acids. Alternatively, azido function containing “amino” acids, or other azide containing auxiliaries can be introduced in for instance a peptide-type active group. Also-SH groups present in, for instance, peptide structures can be used to introduce, for example, azide groups.

Dendrimers of the invention containing at their periphery a high number of groups able to form the aromatic heterocyclic bridges in a click reaction can even be used to attach unprotected peptides.

Many azide containing compounds inclusive dendrimers can be prepared by converting an amino moiety into an azide moiety even when other reactive groups are present. In the working examples herein-below an example of such a conversion is shown. Further, examples of azido (di)peptides and biologically relevant azido peptides, such as Leu-enkephalin, antimicrobial peptides, which can easily be converted into the cycloadducts of the present invention will be shown.

The skilled person also knows that acetylene moieties can easily be introduced in particular molecules. Also for this introduction, examples will be given herein-below.

Densely functionalized dendrimers are promising protein mimics.

The process of the present invention, especially when microwave-assisted, leads to an increased efficiency, is very fast and can be used for a variety of active compounds containing an acetylene, azide or nitrile oxide moiety. The click reaction is catalyzed by copper catalysts, and especially copper compounds soluble in the reaction solvent.

The active groups to be used to form the multivalently substituted dendrimers of the invention encompass peptides, including oligopeptides up to entire proteins, carbohydrates and pharmaceutically active drug molecules. Such active groups, and especially multivalent dendrimeric peptides according to the invention can, for instance, be used as synthetic vaccines, for diagnostic purposes or as targeting devices, for example in the treatment of cancer or infections. An example of a chemical probe for the detection of the cancer-linked marker galectin-3 is described in working example 8. This is only an example. Synthetic molecular probes can e.g. tag a particular protein class, typically based on catalytic activity. The advantage of this path is that information about the quantities of functional proteins is obtained. Probes designed for enzymes can covalently capture their target proteins by taking advantage of their catalytic mechanisms with the use of suicide inhibitors. However, this cannot be done for proteins that merely bind their target molecules or for enzymes for which no suicide inhibitors exist. For these cases photoreactive groups have been linked to the ligands in order to attach to nearby protein residues. Such probes can thus capture proteins as a function of their binding activity within a complex sample as was shown for kinases, metalloproteases, HMG-CoA reductase, and aspartic proteases. An example of non-catalytic protein group are the carbohydrate binding lectins. The galectins, a group of 15 β-galactoside binding lectins, are a particularly interesting subgroup thereof due to their involvement in many biological processes. In particular, galectin-3 is expressed widely in epithelial and immune cells and its expression is correlated with cancer aggressiveness, metastasis, and apoptosis. In this context, galectin-3 can be considered an emerging cancer marker. A significant leap can be made in development by detection of galectin-3 in complex cell lysates, due to the introduction of multivalency in order to increase galectin-3 affinity and lower the detection limit. Multivalency is of great importance in the biological mechanism of action of the galectins which often involves cross-linking in aggregation.

In one of the most preferred embodiments, also the “base” of the dendrimer can be substituted, either by an active compound, such as a homing device or targeting moiety, e.g. a peptide, protein or antibody; or by a label, such as a fluorescent label; by an isotope chelator; by a drug; or by an adjuvant. More in detail, at the “base” or “core” of the dendrimer, a functional group may be present that can be used for attachment of a functional group.

By a suitable combination of substitutions at the periphery and at the base, the present invention renders it possible to achieve a wide variety of applications.

For example, if the base of the dendrimer contains a fluorescent label or a chelator, and the periphery contains a number of peptides or carbohydrates, valuable diagnostics or imaging reagents can be provided. If for instance a radio isotope is present, this may be used for selective in vivo irradiation; if a stable isotope is present, this may be used for in vivo MRI, etc. Also a drug compound can be targeted to a particular site in vivo to achieve a very selective application of the drug, which is, e.g., highly suitable to treat certain types of cancer. In these embodiments, the substituted periphery of the dendrimer effects the desired targeting.

Alternatively, it is also possible to have the targeting moiety, such as an antibody, attached to the base of the dendrimer to escort a dendrimer containing a high number of, e.g., anti-cancer drugs to a selected site in a system.

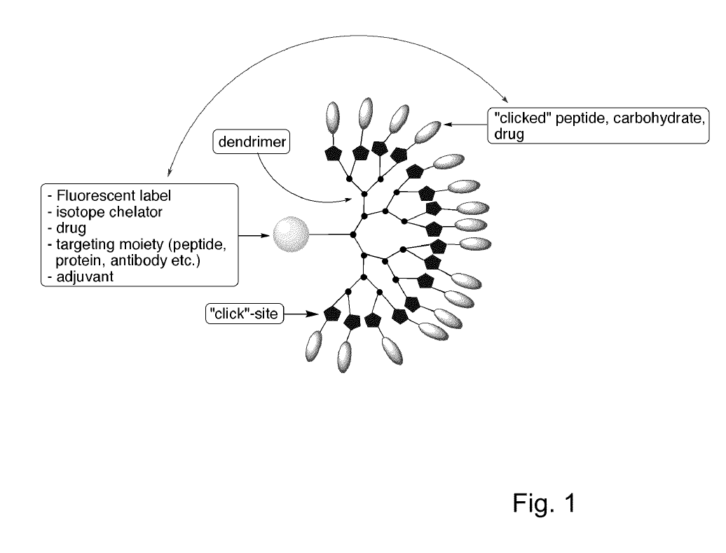

FIG. 1 shows a schematic representation of a multivalent dendrimer according to the invention.

The present invention will now be described in more detail, while referring to the following, non-limiting examples. Unless otherwise indicated, percentages are percentages by weight drawn to the weight of the total product.

In these examples, the design, synthesis and biological evaluation of dendrimeric carbohydrates will be shown. More in detail, it will be shown that peptides can be efficiently attached to a derivatized version of the earlier described (see the article of Brouwer et al. referred to herein-above) dendrimers using a 1,3-dipolar cycloaddition (“click”-chemistry), which was conveniently assisted by microwave irradiation to ensure a complete modification of the alkyne end groups.

The synthesis will be described of the required dendrimeric alkynes used in the attachment of azido peptides either derived from the α-amino group, -amino group of lysine or an ω α-aminohexanoyl spacer to obtain di-, tetra-, octa- and hexadecavalent dendrimeric peptides.

### INTRODUCTION TO THE EXAMPLES

The peptides used in the examples were synthesized on an Applied Biosystems 433 A Peptide Synthesizer.

Analytical HPLC runs were carried out on a Shimadzu HPLC system and preparative HPLC runs were performed on a Gilson HPLC workstation. Analytical HPLC runs were performed on Alltech Adsorbosphere XL C18 and Alltech Prosphere C4 columns (250×4.6 mm, pore size 300 Å, particle size: 5 μm) or on a Merck LiChroCART CN column (250×4.6 mm, pore size 100 Å, particle size 5 μm) at a flow rate of 1.0 mL/min using a linear gradient of buffer B (0-100% in 25 min) in buffer A (buffer A: 0.1% TFA in H₂O buffer B: 0.1% TFA in CH₃CN/H₂O 95:5 v/v). Preparative HPLC runs were performed on an Alltech Prosphere C4 column (250×22 mm, pore size 300 Å, particle size 10 μm) or on a Merck LiChroCART CN column (250×10 mm, pore size 100 Å, particle size: 10 μm) at a flow rate of 4.0 mL/min using a linear gradient of buffer B (0.100% in 50 min) in buffer A (buffer A: 0.1% TFA in H₂O, buffer B: 0.1 TFA in CH₃CN/H₂O 95:5 v/v.

Liquid chromatography electrospray ionization mass spectrometry was measured on a Shimadzu LCMS-QP8000 single quadrupole bench-top mass spectrometer operating in a positive ionization mode.

MALDI-TOF analysis was performed on a Kratos Axima CRF apparatus with bradykinin (1-7) monoisotopic [M+H]⁺757.399), human ACTH (18-39) (monoisotopic [M+H]⁺2465.198) bovine insulin oxidized B chain (monoisotopic [M+H]⁺3494.651), bovine insulin (monoisotopic [M+H]⁺5730.609) and equine cytochrome c (average [M+H]⁺12361.96) as external references and α-cyano-4-hydroxycinnamic acid or sinapic acid as matrices.

¹H-NMR spectra were recorded on a Varian G-300 (300 MHz) spectrometer and chemical shifts are given in ppm (δ) relative to TMS. ¹³C-NMR spectra were recorded on a Varian G-300 (75.5 MHz) spectrometer and chemical shifts are given in ppm (δ) relative to CDCl₃ (77.0 ppm). The ¹³C-NMR spectra were recorded using the attached proton test (APT) sequence. R_{f} values were determined by thin layer chromatography (TLC) on Merck precoated silicagel 60F254 plates. Spots were visualized by UV-quenching, ninhydrin or Cl₂/TDM¹. Elemental analyses were done by Kolbe Microanalytisches Labor (Mülheim an der Ruhr, Germany).

The general procedure for the microwave-assisted click reaction: the alkyne (1 equiv.) and the azide (1.3 equiv. per arm) were dissolved in 3 mL DMF/H₂O 1:1 v/v. To this solution, CuSO₄.5H₂O (0.05 equiv.) and Na-ascorbate (0.50 equiv.) were added. The reaction mixture was placed in a microwave reactor (Biotage) and irradiated during 5-30 min. at 100° C. The cycloaddition reaction was monitored on TLC for completion of the reaction.

### Example 1

The approach described by Brouwer et al. for the convergent synthesis of amino acid based dendrimers was adapted in order to obtain dendrimers with surface propargyl groups to enable a 1,3-dipolar cycloaddition (“click”) reaction with amino acid/peptide derived azides.

First generation dendrimer 1 was synthesized from 3,5-dihydroxy-benzoic acid in an overall yield of 81% as shown in Scheme 1.

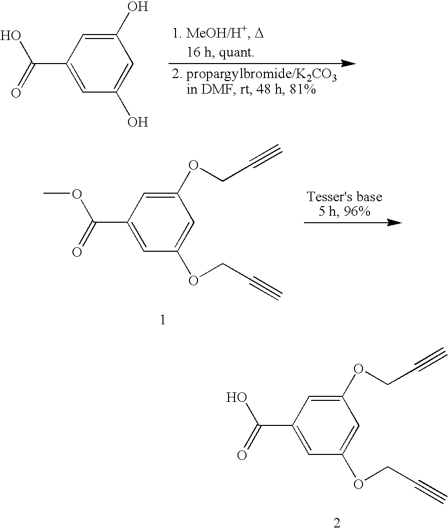

In detail, 3,5-dihydroxymethylbenzoate (21.4 g, 130 mmol) was dissolved in dry DMF (250 mL) and anhydrous K₂CO₃ (45 g, 330 mmol, 2.5 equiv.) was added. To this suspension, a solution of propargylbromide in toluene (35 mL, 314 mmol, 2.5 equiv.) was added dropwise. The reaction mixture was stirred for 48 h at room temperature. Then, DMF was removed by evaporation and the residue was redissolved in EtOAc (400 mL) and the organic phase was washed with H₂O.

(3×100 mL) in KHSO₄ (3×100 mL) and brine (3×100 mL), dried (Na₂SO₄) and evaporated in vacuo. The residue was recrystallized from EtOAc/hexane to obtain 1 as off-white crystals in 81% yield (25.2 g). R_{f}(EtOAc/hexane 4:1 v/v): 0.76; R_{f} (DMC/MeOH 98:2 v/v): 0.87; R_{f}(CHCl₃/MeOH/AcOH 95:20:3 v/v): 0.83; ¹H-NMR (CDCl₃) δ 2.55 (t (J 2.47 Hz), 2H), 3.91 (s, 3H), 4.72 (d (J 2.47 Hz), 4H), 6.81 (t (J 2.20 Hz), 1H), 7.29 (d (J 2.20 Hz), 2H); ¹³C-NMR (CDCl₃) δ 52.4, 56.0, 76.0, 77.9, 107.5, 108.8, 132.0, 157.8, 158.4; elemental analysis: calculated for C₁₄H₁₂O₄ C, 68.83; H, 4.95. found C, 68.76; H, 4.95.

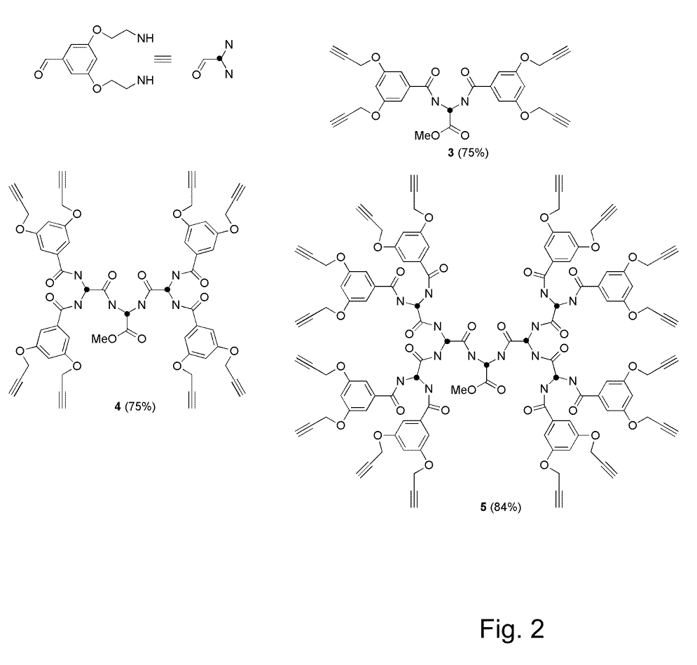

Methyl ester 1 was saponified with Tesser's base (Tesser and Balvert-Geerts, Int. J. Peptide Protein Res. (1975) 7:295) to obtain acid 2 and was subsequently used in the synthesis of the second, third and fourth generation dendrimer 3, 4 and 5, respectively in 75-84% yields (FIG. 2).

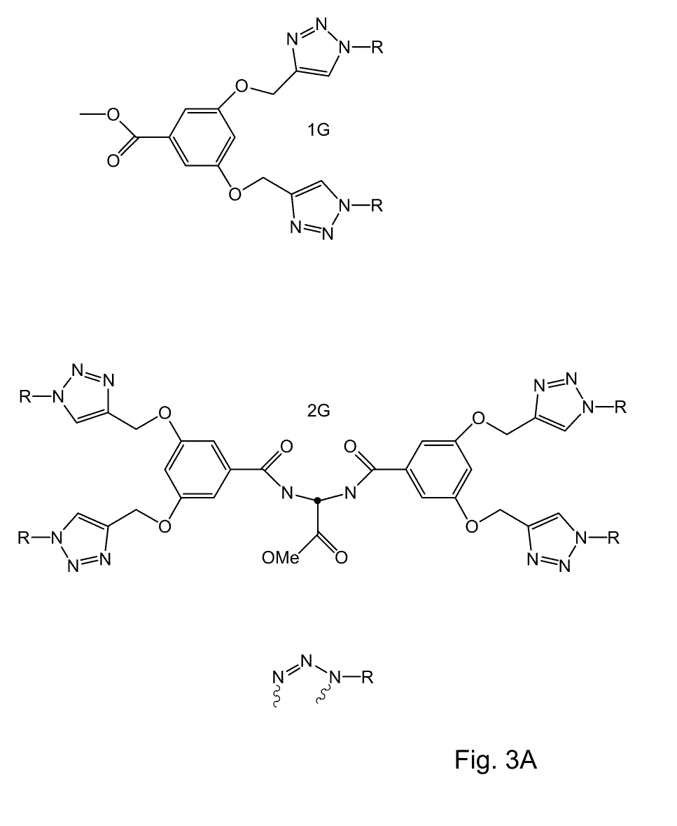

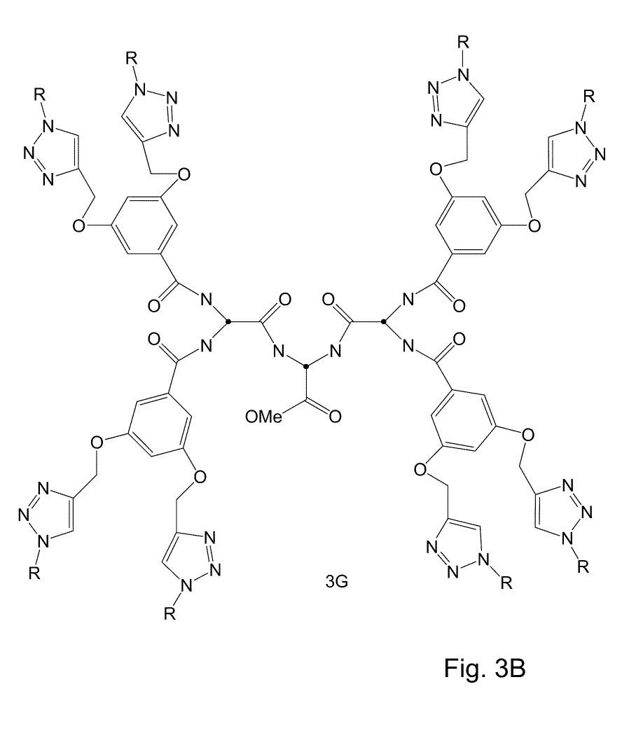

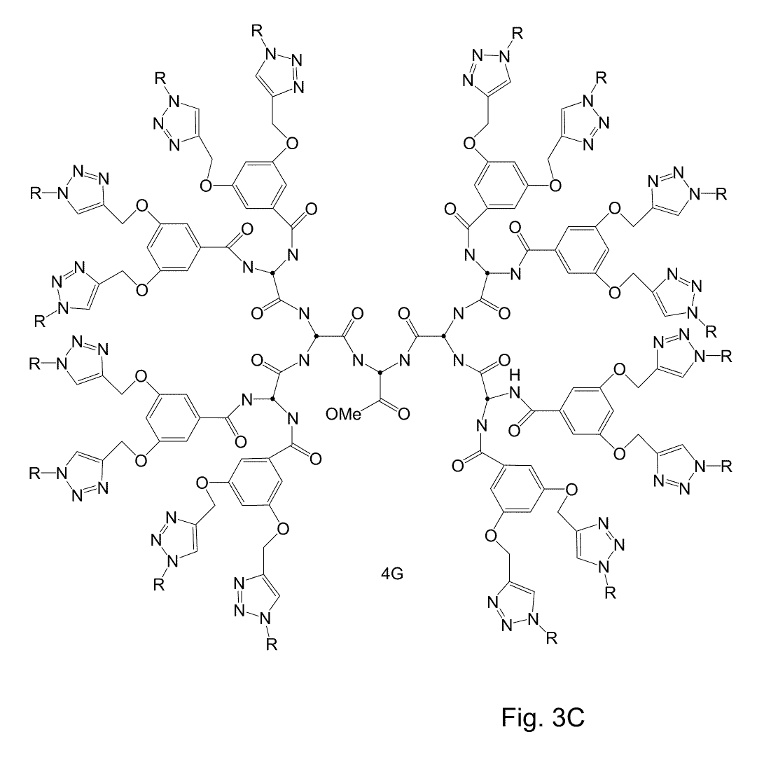

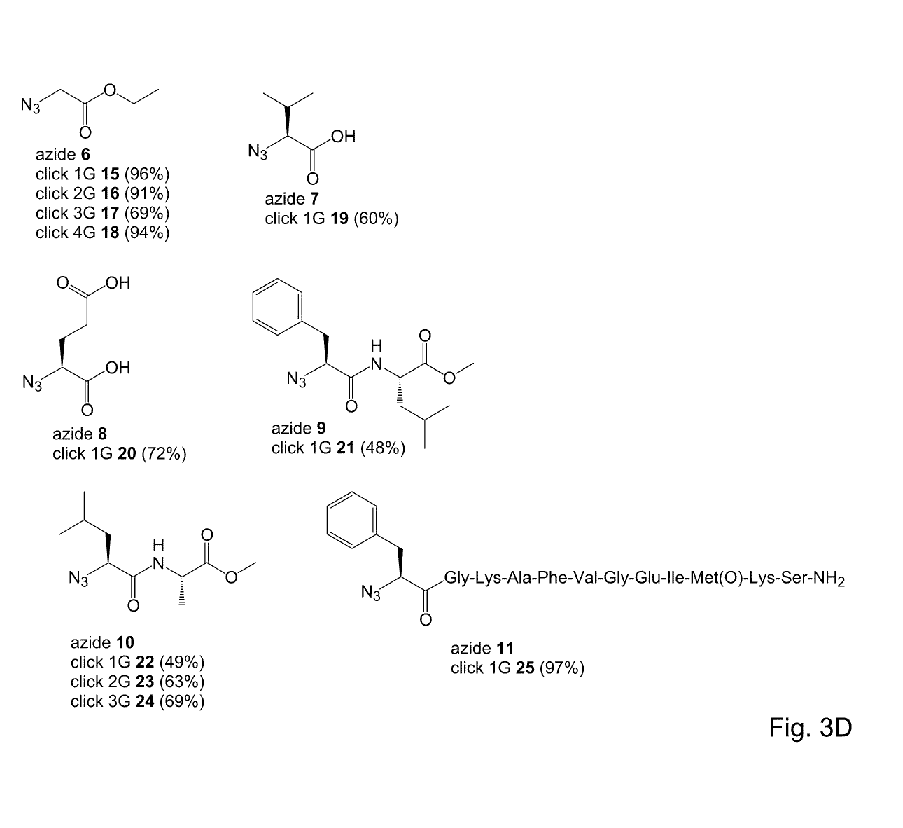

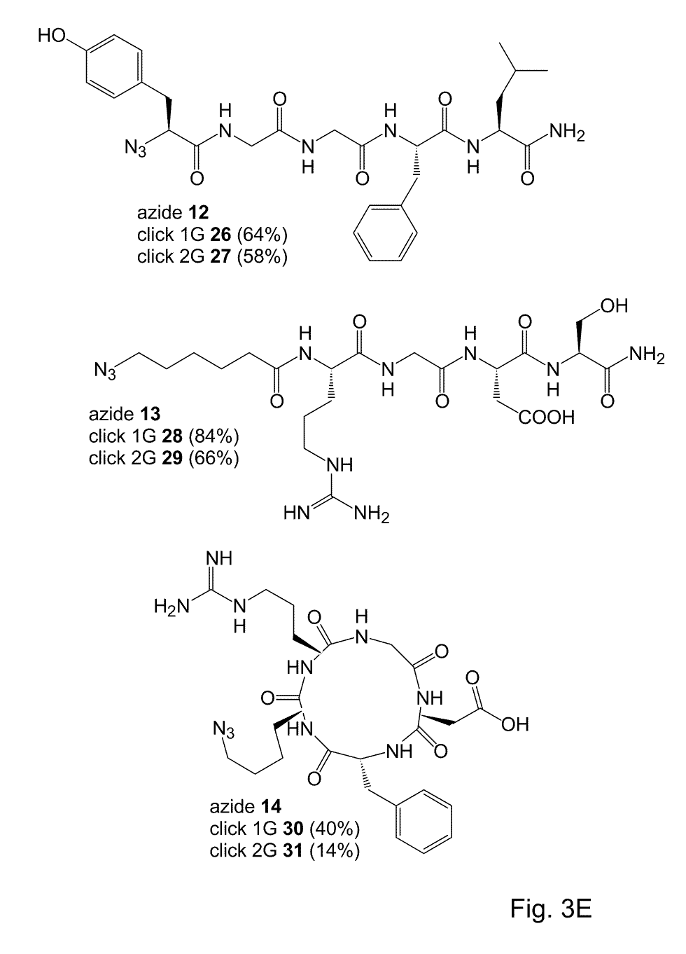

The azido peptides 6-14 used were synthesized according to literature procedures (Azide 6 was prepared according to: S. G. Alvarez and M. T. Alvarez, Synthesis, (1997), 413; Azides 7-10, 13 and 14 were synthesized according to: J. T. Lundquist, IV and J. C. Pelletier, Org. Lett. (2001) 3:781; azido peptides 11 and 12 were synthesized according to: D. T. S. Rijkers et al., Tetrahedron Lett., (2002) 43:3657) (FIG. 3).

Azide 6 was mixed with acetylene 1 in the presence of CuSO₄/Na-ascorbate/Cu-wire in different solvent systems for 16 h at room temperature. Monitoring by TLC showed that formation of the monovalent cycloadduct proceeded rapidly, and conversion to the divalent product was sluggish. Despite this 15 was isolated in fair yields (43-56%).

### Example 2

The results of Example 1 were tremendously improved by running the reaction under microwave irradiation. After 10 min at 100° C. using THF/H₂O (1/1) as a solvent in the presence of CuSO₄/Na-ascorbate, 15 was isolated in 93% yield (FIG. 3). A slight further improvement of the yield to 96% was achieved by performing the reaction in aqueous DMF.

Using the former solvent systems, divalent peptide cycloadducts 19-22 (FIG. 3) were obtained in fair to good yields (48-72%).

The lower yields may be explained by the (significant) lower solubility of 19-22 compared to 15.

### Example 3

Amino acid azide 6 and peptide azide 10, respectively, were reacted with second generation dendrimer 3 for 5-10 min at 100° C. Amino acid azide 6 gave a precipitate which was only soluble in DMF or DMSO and was characterized (¹H—, ¹³C-NMR (APT, HMBC and HSQC), ESMS and MALDI-TOF) as tetravalent amino acid cycloadduct 16 and obtained in excellent yield (91%).

In detail, for 16: alkyne 3 (57 mg, 84 gmol, 1 equiv) and azide 6 (70 mg, 545 gmol, 1.6 equiv per arm) were dissolved in THF/H₂O (2 mL, 1/1) and Na-ascorbate (8 mg, 40 gmol, 50 mol-%) followed by CuSO₄.5H₂O (1 mg, 4 gmol, 5 mol-%) were added. The reaction mixture was placed in the microwave reactor (Biotage) and irradiated at 100° C. during 5 min. The precipitate was filtered off, washed with ice-cold methanol and dried in a dessicator. Compound 16 was obtained as a white solid in 91% yield. R_{f}(CHCl₃/MeOH/AcOH 95/20/3): 0.68; ¹H-NMR (300 MHz, DMSO-d₆): δ 1.21 (t, 12H, J=7.14 Hz), 3.59 (m, 4H), 3.81 (s, 3H), 4.17 (q, 8H, J=7.14 Hz), 4.21 (m, 4H), 5.21 (s, 8H), 5.42 (s, 8H), 6.82 (m, 1H), 6.91 (m, 2H), 7.09 (m, 2H), 7.15 (m, 4H), 8.24 (s, 4H), 8.63 (t, 2H); ¹³C-NMR (75 MHz, DMSO-d₆): δ 14.2, 40.5, 50.6, 52.5, 61.5, 61.7, 66.6, 104.5, 106.6, 107.8, 126.3, 131.8, 136.5, 142.7, 159.2, 159.9, 166.1, 167.4; MALDI-TOF: calculated for C₅₄H₆₂N₁₄O₁₈: 1194.437, found: [M+H]⁺1195.597, [M+Na]⁺1217.578; elemental analysis calculated for C₅₄H₆₂N₁₄O₁₈: C, 54.27%; H, 5.23%; N, 16.41%, found: C, 54.16%; H, 5.17%; N, 16.22%.

Tetravalent peptide cycloadduct 23 was isolated by extraction with EtOAc and obtained in a fair yield of 63% after crystallization from EtOAc/hexane.

Using microwave irradiation, octavalent peptide 24 (48%) as well as octavalent and hexadecavalent systems 17 and 18 were prepared in 69 and 94% yield, respectively (FIG. 3).

### Example 4

In this example the attachment of larger or bioactive peptides to these dendrimeric systems using the microwave-assisted cycloaddition chemistry is described. Particularly, azido peptide 11, representing the amino acid residues 12-23 of the antimicrobial peptide magainin I amide (Zasloff, Proc. Natl. Acad. Sci. USA (1987) 84:5449) was coupled under microwave assistance and divalent cycloadduct 25 was obtained in nearly quantitative yield (FIG. 3). Work-up of this water-soluble divalent peptide was easy as well as purification by HPLC or size exclusion chromatography.

Then, azido-Leu-enkephalin (12), azide 13 (a fibronectin active fragment, connected to an ω α-amino hexanoic acid spacer) and the cyclo RGD azido peptide 14 (an α_{V}β₃ α integrin binding RGD peptide for tumor targeting; Haubner et al., J. Am. Chem. Soc., (1996) 18:7461; Haubner et al., J. Nucl. Med. (2001) 42:326; Janssen et al., Cancer Res., (2002) 62:6146; Thurmshirn et al., Chem. Eur. J. (2003) 9:2717) were coupled to alkynes 2 and 3. The divalent and tetravalent peptides 26, 28, 30 and 27, 29, 31, respectively (FIG. 3) were obtained after purification by HPLC in yields ranging from 14 to 84%. These peptides were identified by MALDI-TOF analysis.

In addition, the coupling of 4^{th} generation dendrimer (16 end-groups) 5 with cyclo RGD azido peptide 14 is illustrated in order to obtain a hexadecavalent peptide dendrimer. HPLC analysis after work-up showed that the starting materials were consumed. By SDS-PAGE, bands were visible corresponding to a molecular weight ranging between 53000-65000 Da, corresponding to an aggregate in a tetra- or pentamer.

For the compounds identified in Examples 1-4, the following analytical data have been obtained:

Compound 2: ¹H-NMR (DMSO-d₆) δ 2.50 (broad s, 2H), 4.85 (d (J 2.20 Hz), 4H), 6.86 (t (J2.47 Hz), 1H), 7.17 (d (J2.47 Hz), 2H).

Compound 3: R_{f}(EtOAc/hexane 4:1 v/v): 0.03; R_{f}(CHCl₃/MeOH/AcOH 95:20:3 v/v): 0.80; ¹H-NMR (CDCl₃) δ 2.55 (t (J 2.47 Hz), 4H), 3.83 (m, 4H), 3.88 (s, 3H), 4.07 (t (J 4.94 Hz), 4H) 4.68 (d (J 2.47 Hz), 8H), 6.54 (t (J 2.20 Hz), 1H), 6.72 (t (J 2.20 Hz), 2H), 6.93 (t (J 5.77 Hz), 2H), 7.05 (d (J 2.47 Hz), 4H), 7.09 (d (J 2.47 Hz), 2H); ¹³C-NMR (CDCl₃) δ 40.4, 53.2, 56.9, 57.6, 77.0, 78.8, 106.3, 107.1, 107.6, 109.0, 132.9, 137.4, 159.6, 160.3, 167.4, 168.1; MS analysis: calculated for C₃₈H₃₄N₂O₁₀ 678.22, found ES-MS 679.40 [M+H]⁺, 701.45 [M+Na]⁺; MALDI-TOF 679.298 [M+Na]⁺.

Compound 4: R_{f}(CHCl₃/MeOH/AcOH 95:20:3 v/v): 0.73; ¹H-NMR (DMSO-d₆) δ 2.50 (broad s, 8H), 3.59 (m, 12H) 3.61 (s, 3H)m 4.14 (m, 12H)m 4.83 (d, 8H), 6.78 (m, 7H), 7.12 (m, 14H), 8.68 (m, 6H); ¹³C-NMR (DMSO-d₆) δ 37.8, 49.8, 53.3, 63.7, 76.0, 76.4, 102.5 103.4, 104.2, 105.1, 129.1, 133.8, 155.7, 156.9, 157.1, 163.3; MS analysis: calculated for C₈₆H₇₈N₆O₂₂ 1546.52, found MALDI-TOF 1547.490 [M+H]⁺, 1569.496 [M+Na]⁺.

Compound 5: ¹H-NMR (DMSO-d₆) δ 2.50 (broad s, 16H), 3.58 (m, 28H), 3.81 (s, 3H), 4.13 (m, 28H), 4.82 (d, 32H), 6.88 (m, 16H), 7.11 (m, 30H), 8.70 (m, 14H): MS analysis: calculated for C₁₈₂H₁₆₆N₁₄O₄₆ 3282.33, found MALDI-TOF 3321.467 [M+K]⁺.

Compound 6: ¹H-NMR (CDCl₃) δ 1.32 (t (J 7.14 Hz), 3H), 3.88 (s, 2H), 4.26 (q (J 7.14 Hz), 2H); ¹³C-NMR (CDCl₃) δ 14.0, 50.2, 61.8, 168.2.

Compound 7: ¹H-NMR (CDCl₃) δ 1.01/1.03-1.06/1.08 (dd J 15 11 Hz, J=6.88 Hz), 6H), 2.23 (m, 1H), 3.79 (d J 649 Hz), 1H.

Compound 8: ¹H-NMR (CDCl₃) δ 2.09/2.23 (double m, 2×1H), 2.59 (m, 2H), 4.13 (m, 1H).

Compound 9: ¹H-NMR (CDCl₃) δ 0.85/0.88 (dd J 6.59 Hz, J=1.10 Hz), 6H), 1.26-1.57 (broad m, 3H), 3.02/3.05-3.07/3.09 (dd J 14.1 Hz, J=7.5 Hz), 1H), 3.28/3.32/3.34 (dd J 14.1 Hz, J=4.2 Hz), 1H), 3.73 (s, 3H), 4.28 (m, 1H), 4.52 (m, 1H), 6.21 (broad s, 1H), 6.65 (d J 8.52 Hz), 1H), 7.29 (m, 5H); ¹³C-NMR (CDCl₃) δ 22.0, 23.0, 24.8, 38.5, 41.5, 50.8, 52.6, 65.5, 127.5, 128.8, 129.8, 136.1, 168.4, 173.1.

Compound 10: ¹H-NMR (CDCl₃) δ 0.96/0.97-0.98/0.99 (dd J 6.4 Hz, J=2.3 Hz), 6H), 1.42 (d J 7.14 Hz), 3H), 1.67-1.85 (broad m, 3H), 3.78 (s, 3H), 3.97 (m, 1H), 4.57 (m, 1H), 6.89 (d, 1H).

Compound 11: Synthesized as described, see reference 3: [M+H]⁺; calculated 1338.70, found 1338.72 (ES-MS).

Compound 12: Rₜ; 17.88 min (C4); Rₜ: 19.66 min (C18); MS analysis: calculated for C₂₈H₃₆N₈O₆ 580.27, found ES-MS 581.55 [M+H]⁺, 603.55 [M+Na]⁺.

Compound 13: Rₜ: 12.89 min (C4); Rₜ: 15.57 min (C18); MS analysis: calculated for C₂₁H₃₇N₁₁O₈ 571.28, found 572.55 [M+H]⁺.

Compound 14: Rₜ: 16.81 min (C4); MS analysis; calculated for C₂₇H₃₉N₁₁O₇ 629.30 found 630.55 [M+H]⁺, 652.70 [M+Na]⁺, 668.25 [M+K]⁺.

Compound 15: R_{f}:(CHCl₃/MeOH/AcOH 95:20:3 v/v): 0.73; ¹H-NMR (CDCl₃) δ 1.28 (t J 7.14 Hz), 6H), 3.89 (s, 3H), 4.24 (q J 7.14 Hz), 4H), 5.19 (s, 4H), 5.19 (s, 4H), 5.21 (s, 4H), 6.81 (m, 1H), 7.27 (m, 2H), 7.81 (s, 2H); ¹³C-NMR (CDCl₃) δ 14.0, 33.8, 50.8, 52.3, 62.0, 62.4, 106.9, 108.6, 124.3, 132.1, 143.9, 159.1, 166.6; Ms analysis: calculated for C₂₂H₂₆N₆O₈ 502.48 found ES-MS 503.30 [M+H]⁺, 525.30 [M+Na]⁺; MALDI-TOF 503.259 [M+H]⁺.

Compound 16: R_{f}: (CHCl₃/MeOH/AcOH 95:20:3 v/v); 0.68 ¹H-NMR (DMSO-d₆) δ 1.21 (t J 7.14 Hz), 12H), 3.59 (m, 4H), 3.81 (s, 3H), 4.17 (q J 7.13 Hz), 8H), 4.21 (m, 4H), 5.21 (s, 8H), 5.42 (s, 8H), 6.82 (m, 1H), 6.91 (m, 2H), 7.09 (m, 2H), 7.15 (m, 4H), 8.24 (s, 4H), 8.63 (t, 2H); ¹³C-NMR (DMSO-d₆) δ 14.2, 40.5, 50.6, 52.5, 61.5, 61.7, 66.6, 104.5, 106.6, 107.8, 126.3, 131.8, 136.5, 142.7, 159.2, 159.9, 166.1, 167.4; MS analysis: calculated for C₅₄H₆₂N₁₄O₁₈ 1194.437, found MALDI-TOF 1195.597 [M+H]⁺, 1217.578 [M+Na]⁺; elemental analysis: calculated for C₅₄H₆₂N₁₄O₁₈ C, 54.27%; H, 5.25%; N, 16.41%. found C, 54.16%; H, 5.17%; N, 16.22%.

Compound 17: ¹H-NMR (DMSO-d₆) δ 1.20 (t J 7.14 Hz), 24H), 3.60 (broad, 12H), 3.79 (s, 3H), 4.18 (m, 28H), 5.21 (s, 16H), 5.41 (s, 16H), 6.72 (m, 2H), 6.81 (m, 1H), 6.93 (m, 4H), 7.04 (m, 6H), 7.18 (m, 8H), 8.23 (s, 8H), 8.68 (m, 6H), MS analysis: calculated for C₁₁₈H₁₃₄N₃₀O₃₈ 2580.50, MALDI-TOF 2581.012 [M+H]⁺, 2603.116 [M+Na]⁺; elemental analysis: calculated for C₁₁₈H₁₃₄N₃₀O₃₈ C, 54.8%; H, 5.37%; N, 16.00%. found C, 54.88%; H, 5.17%; N, 16.19%.

Compound 18: MS analysis: calculated for C₂₄₆H₂₇₈N₆₂O₇₈ 5347.969, found MALDI-TOF 5389.460 [M+CH₃CN)+H]⁺.

Compound 19: Rₜ: 19.05 min (C4) Rₜ: 20.84 min (C18); ¹H-NMR (DMSO-d₆) δ 0.74 (d J 6.59 Hz), 6H), 0.94 (d J 6.59 Hz), 6H), 1.24 (m, 2H), 3.84 (s, 3H), 4.10 (broad s, 2H), 5.03 (m, 2H), 5.21 (s, 4H), 7.07 (m, 1H), 7.18 (m, 2H), 8.30 (s, 2H); ¹³C-NMR (DMSO-d₆) δ 18.5, 19.4, 31.0, 52.6, 61.8, 107.0, 108.3, 124.9, 131.8, 134.2, 142.2, 159.4, 166.1, 170.3; MS analysis: calculated for C₁₆H₃₀N₆O₈ 530.21, found MALDI-TOF 531.339 [M+H]⁺, 553.308 [M+Na]⁺.

Compound 20: Rₜ: 15.98 min (CN); MS analysis: calculated for C₂₄H₂₆N₆O₁₂ 590.16, found ES-MS 591.29 [M+H]⁺.

Compound 21: ¹H-NMR (CDCl₃) δ 0.85 (d J 5.49 Hz), 12H), 1.55 (m, 6H), 3.38 (m, 2H), 3.57 (m, 2H), 3.67 (s, 6H), 3.90 (s, 3H), 4.49 (m, 2H), 5.13 (s, 4H), 5.57 (m, 2H), 6.77 (m, 1H), 6.99 (m, 4H), 7.27 (m, 6H), 7.33 (m, 6H), 7.86 (s, 2H); ¹³C-NMR (CDCl₃) δ 21.6, 22.5, 24.6, 39.5, 40.8, 51.1, 52.2, 61.9, 65.4, 106.8, 108.4, 123.7, 127.2, 128.5, 128.7, 132.0, 134.9, 143.4, 159.0, 166.2, 167.2, 172.3; MS analysis: calculated for C₄₆H₅₆N₈O₁₀ 880, found ES-MS 881.50 [M+H]⁺, 903.30 [M+Na]⁺; MALDI-TOF 881.288 [M+H]⁺, 903.244 [M+Na]⁺; elemental analysis: calculated for C₄₆H₅₆N₈O₁₀ C, 62.71%; H, 6.41%; N, 12.72%. found C, 62.64%; H, 6.37%; N, 12.64%.

Compound 22: ¹H-NMR (DMSO-d₆) δ 0.89 (m, 12H), 1.24 (m, 2H), 1.30 (d J 7.42 Hz), 6H), 1.89 (broad m, 4H) 3.64 (s, 6H), 3.84 (s, 3H), 4.25 (m, 2H), 5.19 (s, 4H), 5.47 (m, 2H) 7.05 (m, 1H), 7.19 (m, 2H), 8.35 (s, 2H), 9.03 (d J 6.59 Hz), 2H), ¹³C-NMR (DMSO-d₆) δ 17.3, 22.2, 23.0, 24.8, 41.3, 48.5, 52.7, 53.0, 61.4, 62.2, 107.4, 108.7, 132.3, 143.0, 159.9, 166.5, 168.7, 173.3; MS analysis: calculated for C₃₄H₄₈N₈O₁₀ 728.35, found ES-MS 729.55 [M+H]⁺, 751.45 [M+Na]⁺; MALDI-TOF 729.417 [M+H]⁺, 751.3598 [M+Na]⁺; elemental analysis: calculated for C₃₄H₄₁N₄O₇ C, 56.031%; H, 6.64%; N, 15.38%. found C, 56.10%, H, 6.60%; N, 15.28%.

Compound 23: ¹H-NMR (CDCl₃) δ 0.87/0.90 (d J 6.59 Hz), 24H), 1.29/1.31 (d J 7.14 Hz), 12H), 1.39 (m, 4H), 2.04 (m, 8H), 3.72 (s, 12H), 3.79 (s, 3H), 3.84 (m, 4H), 4.17 (m, 4H), 4.51 (m, 4H), 5.02 (s, 8H), 5.49 (m, 4H), 6.63 (m, 2H), 6.75 (m, 1H), 6.98 (m, 4H), 7.16 (m, 2H), 7.42 (d J 7.78 Hz), 4H), 7.68 (m, 2H), 8.07 (s, 4H); MS analysis: calculated for C₇₈H₁₀₆N₁₈O₂₂ 1646.77, found MALDI-TOF 1647.730 [M+H]⁺, 1669.732 [M+Na]⁺.

Compound 24: ¹H-NMR (DMSO-d₆) δ 0.76 (m, 48H), 1.21 (m, 8H), 1.28 (d J 7.14 Hz), 24H), 1.98 (m, 16H), 3.58 (overlapping signals 36H), 3.80 (overlapping signals 15H), 4.13 (m, 8H), 4.82 (broad s, 16H), 5.19 (m, 8H), 6.77 (m, 21H), 7.05 (m, 14H), 8.19 (s, 8H); ¹³C-NMR (DMSO-d₆) δ 16.8, 21.7, 22.5, 24.4, 40.5, 48.0, 52.2, 56.0, 66.4, 78.7, 79.1, 99.7, 104.2, 105.2, 106.2, 107.8, 131.8, 136.5, 142.5, 158.4, 159.2, 159.6, 166.0, 166.1, 168.2, 172.7.

Compound 25: Rₜ: 19.1 min (C4); MS analysis: calculated for C₁₃₆H₂O₂N₃₄O₃₆S₂ 2952.872, found MALDI-TOF 2593.310 [M+H]⁺.

Compound 26: MS analysis: calculated for C₇₀H₈₄N1₆O₁₆ 1404.625, found MALDI-TOF 1427.822 [M+Na]⁺.

Compound 27: MS analysis: calculated for C₁₅₀H₁₇₈N₃₄O₂₄ 2999.325, found MALDI-TOF 3021.827 [M+H]⁺.

Compound 28: MS analysis: calculated for C₅₆H₈₆N₂₂O₂₀ 1386.639, found MALDI-TOF 1386.638[M+H]⁺.

Compound 29: Rₜ: 16.78 min (CN); MS analysis: calculated for C₁₂₂H₁₈₂N₄₆O₄₂ 2963.352, found MALDI-TOF 2963.805 [M+H]⁺.

Compound 30: Rₜ: 21.23 min (CN); MS analysis: calculated for C₁₄₆H₁₉₀N₄₆O₃₈ 3195.435, found MALDI-TOF 3195.730 [M+H]⁺.

### Example 5

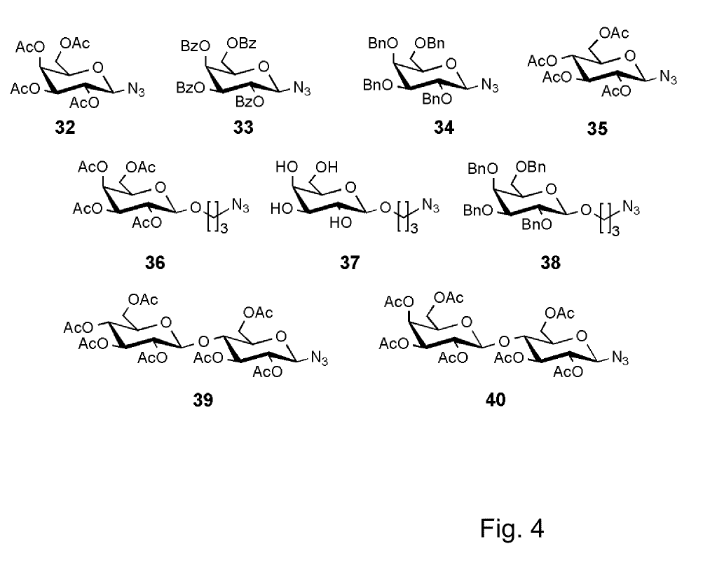

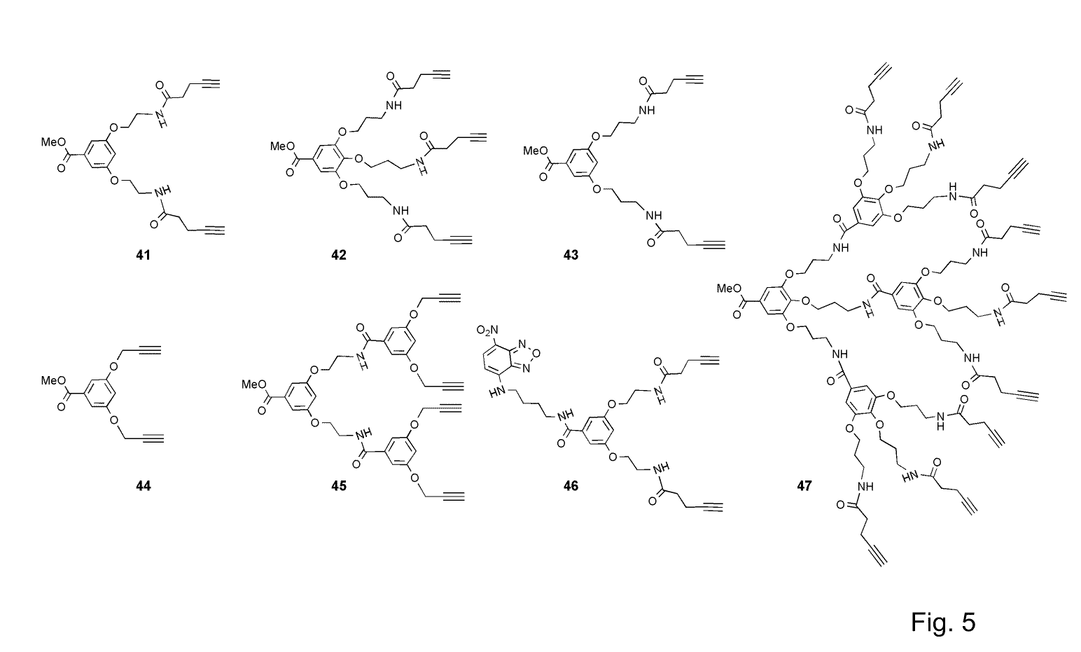

Azido carbohydrates 32-40 of different character, functionalization pattern and reactivity (FIG. 4) were subjected to the cycloaddition reaction with dendrimers 41-47 (FIG. 5) and 19 different triazole glycodendrimers were obtained in good yields. The cycloaddition reaction is depicted in scheme 2:

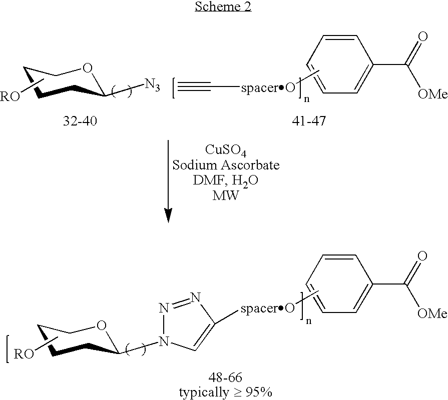

Furthermore, the use of unprotected carbohydrates was demonstrated as well as the straightforward incorporation of a fluorescent label, relevant for biological evaluation.

The carbohydrate derivatives included the monosaccharide galactose that was used without 32-34, or with 36-38, a spacer to the azido function. Its hydroxyl protecting groups were also varied in order to evaluate this parameter. Besides galactose, the monosaccharide glucose (35), the disaccharides cellobiose (39) and lactose (40) were included. The anomeric glycosyl azides were prepared from their respective counterparts by reaction with HBr in AcOH under microwave irradiation. The resulting glycosyl bromides were treated with trimethylsilyl azide and tetrabutyl ammonium fluoride, again using microwave irradiation, which gave the desired peracetylated β-azidosugars for the [3+2]cycloaddition reactions in high yields.

Particularly for the (38) cellobiose example a solution of per-acetylated cellobiose (500 mg), HBr (5 quiv.) and acetic anhydride (4 equiv.) in dry CH₂Cl₂ (10 mL) was exposed to microwave irradiation under sealed vessel conditions at 800° C. for 20 min, after which it was concentrated. The residue was dissolved in dry THF (10 mL) and TMSN₃ (5 equiv.) were added. The mixture was exposed to microwave irradiation at 800° C. for 15 min, then concentrated and purified on a silica column. Elution with toluene-EtOAc 30:1-5:1 gave 38 in 99% yield.

The azido carbohydrates were reacted with di- and trivalent first generation dendrimers (41-44), one tetravalent second generation dendrimer (45), one nonavalent second generation dendrimer (47), as well as a divalent dendrimer with an NBD-fluorescent label (46). The dendritic main structures were prepared using the approach of Example 1, and these amino acid dendrimers were readily synthesized in high yields. Their carbohydrate conjugates were shown to be effective multivalent scaffolds, increasing the binding potencies of their attached carbohydrates by several orders of magnitude.

The copper catalyzed coupling of the azido carbohydrates to divalent alkyne-linked dendrimeric structures involved overnight stirring at room temperature. This yielded a slow conversion to the mono-coupled product. Optimization of the reaction conditions using a microwave reactor (800° C.; 20 min.) and CuSO₄ and sodium ascorbate as the copper(I) source led to formation of the desired triazole glycodendrimers in high yields (Table 1, particularly the dendrimer (30-100 gmol) and the azido carbohydrate (1.5 equiv. per alkyne group), CuSO₄ (30 mol %), and sodium ascorbic acid (60 mol %), were dissolved in DMF (1-1.5 mL) containing several drops of H₂O. The solution was exposed to microwave irradiation at 80° C. for 20 min, then concentrated and purified on a silica column eluting first with CH₂Cl₂-EtOAc 6:1 to recover the excess of starting azido carbohydrate followed by elution with CH₂Cl₂-MeOH 6:1 to obtain the triazole-linked glycodendrimer in the indicated yields.).

The use of an excess (1-5 equiv.) of the azido carbohydrate drives the reactions to completion, while the excess could be readily recovered by column chromatography. The relative low product yields in the reactions with unprotected glycosyl azide 36 or fluorescent labeled 46 containing the first generation dendrimer reflects the product purification rather than the coupling efficiency. All coupling products were fully characterized by ¹H, ¹³C—, 2D-COSY NMR and MS.

TABLE 1
Microwave assisted copper-(I)-mediated synthesis ^{a} of triazole
glycodendrimers under microwave conditions.

| Entry | Azide | Dendrimer | Valency | Yield ^{b} |
|---|---|---|---|---|
| 1 | 32 | 41 | 2 | 96 |
| 2 | 32 | 42 | 3 | 95 |
| 3 | 32 | 44 | 2 | 97 |
| 4 | 32 | 45 | 4 | 98 |
| 5 | 33 | 46 | 2 | 68 |
| 6 | 34 | 41 | 2 | 95 |
| 7 | 34 | 44 | 2 | 97 |
| 8 | 35 | 41 | 2 | 97 |
| 9 | 37 | 41 | 2 | 73 |
| 10 | 37 | 42 | 3 | 80 |
| 11 | 37 | 44 | 2 | 94 |
| 12 | 38 | 43 | 2 | 95 |
| 13 | 39 | 41 | 2 | 95 |
| 14 | 40 | 41 | 2 | 95 |
| 15 | 40 | 42 | 3 | 91 |
| 16 | 40 | 44 | 4 | 98 |
| 17 | 40 | 45 | 4 | 91 |
| 18 | 40 | 46 | 2 | 86 |
| 19 | 36 | 47 | 9 | 97 |

^{a} Reagent and conditions: alkyne containing dendrimer (1 equiv.), azido carbohydrate (1,5 equiv. per alkyne), CuSO₄ (0-15 equiv. per alkyne), sodium ascorbate (0.3 equiv. per alkyne) DMF/H₂O, MW 80° C., 20 min.
^{b} Yield of isolated product in % by weight.

### Example 6

### GlCNac Click Dendrimers

Di-, tetra- and octavalent GINAc-linked “click” glycodendrimers as depicted herein-below were evaluated as adhesion inhibitors of Candida albicans (strain CBS 562) and compared to the free monosaccharide GlcNAc (N-acetyl glucosamine).

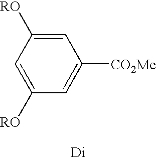

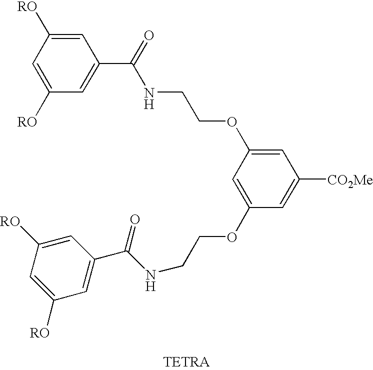

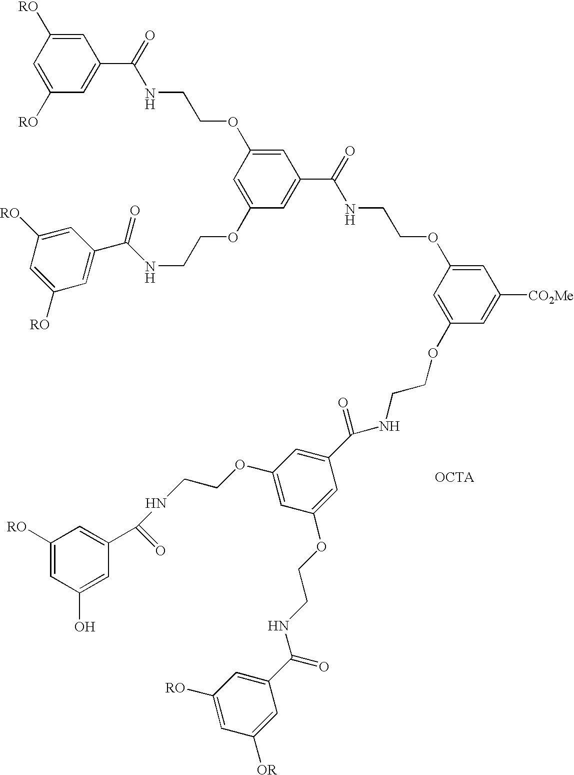

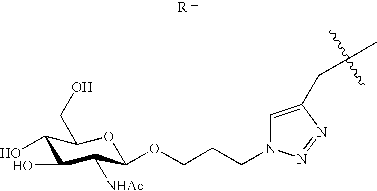

| Compound | Concentration (μM) | Inhibition (%) |
|---|---|---|
| GlcNAc | 113 | 79 |
| Di | 15 | 21 |
| TETRA | 7 | 34 |
| OCTA | 3 | 72 |

From the results it can be shown that in low concentrations already a considerable inhibitive can be obtained. The OCTA-embodiment was very potent.

In addition, as a model for the interference with the natural killer cells of the immune system, the compounds were evaluated in an ELISA type assay for their interaction with the rat receptor NKRPI and the human CD 69 receptor. Large enhancements were observed for the TETRA- and OCTA-embodiments of a factor of more than 100.

| Compound | −log IC₅₀ (NKRPI) | −log IC₅₀ (CD69) |
|---|---|---|
| GlcNAc | 6.0 | 5.3 |
| TETRA | 8.5 | 7.54 |
| OCTA | 8.6 | 7.6 |

### Example 7

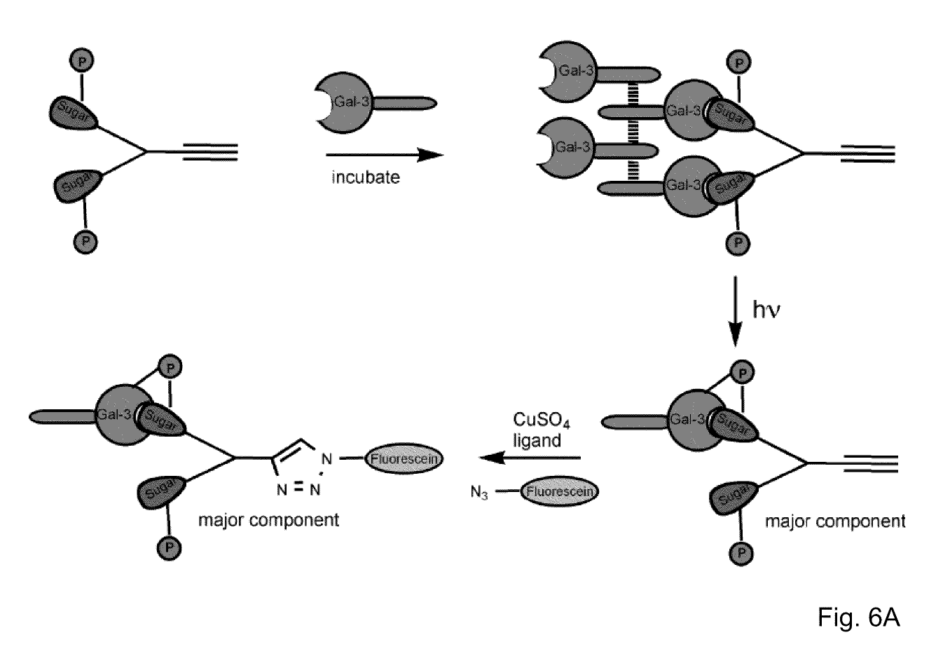

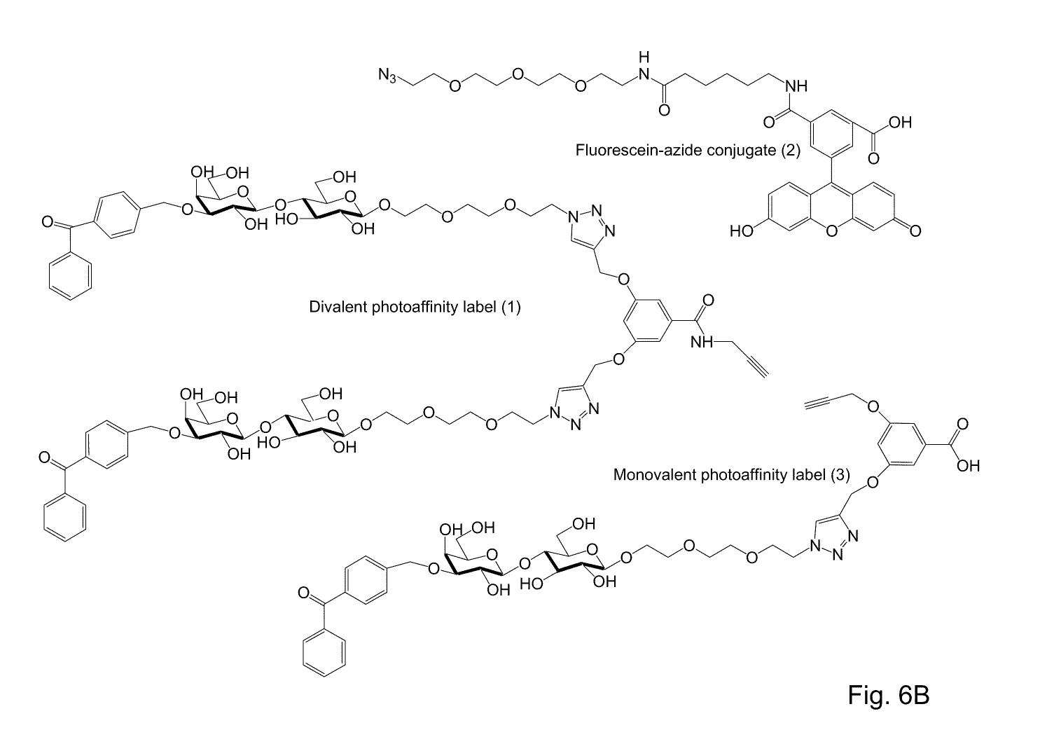

Probe 1 was prepared (see Chart of FIG. 1) with a benzophenone photoaffinity label on the lactose 3′-OH position, where aryl groups are beneficial for binding. The probe was made divalent by linking the previous probe to a divalent scaffold. The alkyne moiety on the probe allows for “click chemistry” to introduce a fluorescent label for visualization (FIG. 6). Introduction of the label in a second step has advantages such as the fewer artifacts due to bulky labels in the crucial photoreaction step. In order to minimize non-selective labelling of proteins by the click reagents, the position of the “click” functional groups was reversed in comparison to the initial study.

To evaluate the probe, protein mixtures were prepared. The first mixture consisted of six common proteins and the second mixture contained a series of seven carbohydrate binding and processing proteins, including several galactose specific ones (protein mix I and II).

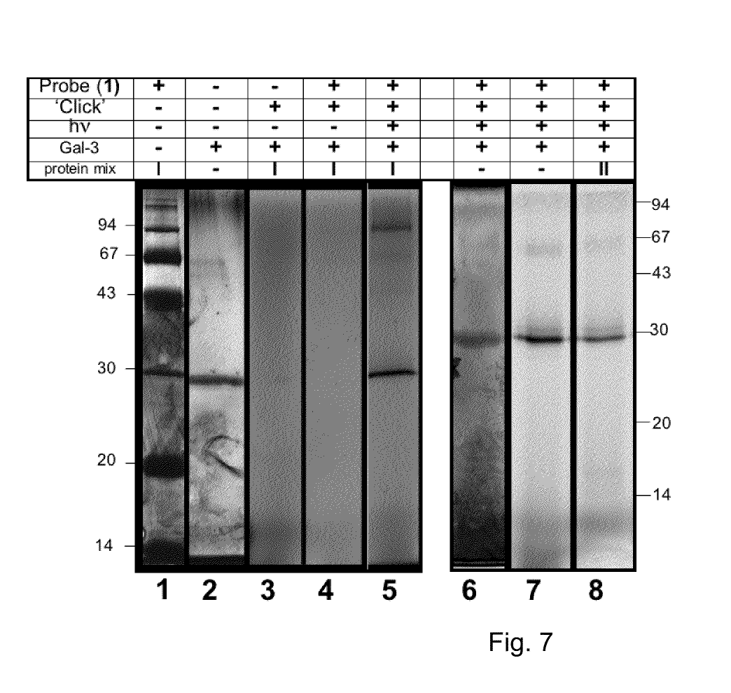

To these mixtures various amounts of galectin-3 were added and also various amounts of probe were applied. Following the above protocol, the fluorescent gel images obtained indicated good selectivity of the probe for galectin-3 (FIG. 7). In the first series some labelling of the very “sticky” albumin was detected. The probe also exhibited selectivity for galectin-3 over galectin-1, under these conditions. A faint band at twice the molecular weight of galectin-3 was seen in a sample only containing galectin-3 (lane 7), indicating the capture of two copies of this protein.

The most favorable probe (1) concentration was found to be 5 μM. At this probe concentration, a two-fold excess of fluorescein-N3 construct 2 was found to be optimal for effective conjugation to the probe galectin-3 adduct. Particularly, these mixes contain the following protein mix 1: α-lactalbumin, trypsin inhibitor, carbonic anhydrase, ovalbumin, albumin and phosphorylase B. Protein mix II: L: α-glucosidase, β-galactosidase, β-glucosidase, galectin-1, peanut agglutinin, Helix aspersa agglutinin, galactosyl transferase. Higher concentrations of 2 gave rise to saturated fluorescence gels and thus poorly visible protein signals. With these concentrations of 1 and 2 the minimal detection limit for galectin-3 was found to be 1-5 ng which is clearly sufficient for application. With the related monovalent probe 3 only 50 ng of galectin-3 could be detected, thus confirming the benefits of multivalency.

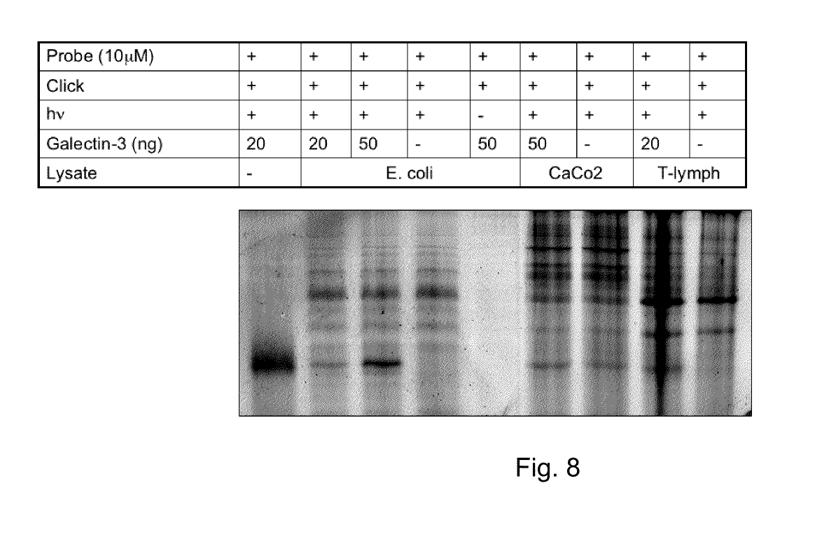

Once the optimal probe concentration and the lower detection limit were known, the methodology was applied to an E. Coli and two human cell line lysates cell to explore the probe sensitivity in a biologically complex environment (FIG. 8). The bacterial lysate was spiked with various concentrations of galectin-3. Following the protocol, fluorescent images of the gel clearly show that galectin-3 can be detected under these conditions. In the corresponding experiments with the CaCo2 lysate, which is known to express galectin-3, both the spiked sample and the non-spiked sample clearly show a band corresponding to the expected galectin-3 position. Similar experiments with a T-lymphocytes cell line, known not to express significant amounts of galectin-3, showed non-spiked sample. While other proteins are also labelled, galectin-3 can be clearly detected reproducibly in cell lysates of biomedical relevance. In order to confirm the presence of endogenous galectin-3 in the CaCo₂ cell line, the presumed galectin-3 band was excised from the gel, digested and analyzed by MS. The analysis confirmed the presence of galectin-3. In conclusion, it is shown the utility of a galectin-3 specific divalent photoaffinity for the visualization of galectin-3 in biological protein mixtures. This facilitates the use of probes in biological galectin-3 studies and may aid in the diagnosis and prognosis of cancers.

1. A multivalent dendrimer, comprising at the dendrimer periphery a number of active compounds, the active compounds being coupled to the dendrimer through a 1,2,3-triazole or 1,2-oxazole heterocyclic bridge.

2. The dendrimer of claim 1, being at least a second generation dendrimer.

3. The dendrimer according to claim 1, being amino acid based.

4. The dendrimer according to claim 1, wherein each active compound at the periphery of the dendrimer is selected from the group consisting of peptides, carbohydrates or drugs.

5. The dendrimer according to claim 1, wherein the active compounds are the same.

6. The dendrimer according to claim 1, wherein the core of the dendrimer is substituted by another active compound than the active compounds at the periphery.

7. A process for the preparation of a multivalent dendrimer according to claim 1, comprising reacting a dendrimer having a number of alkyne moieties at its periphery with active compounds comprising an azide or nitrile oxide moiety, or reacting a dendrimer having a number of azide or nitrile oxide moieties at its periphery with active compounds comprising an alkyne moiety.

8. The process of claim 7, wherein the reaction is microwave assisted.

9. The process of claim 7, wherein the alkyne moiety and/or the azide or nitrile oxide moiety is attached to either the dendrimer or the active compound through a spacer.

10. An amino acid based dendrimer having a number of alkyne moieties at its periphery, or having a number of azide or nitrile oxide moieties at its periphery.

11. Use of the multivalent dendrimer according to claim 1 as a pharmaceutical agent or as a diagnostic agent.
